(19) United States
(12) Patent Application Publication
(10) Pub. No.: US 2023/0366882 A1
(43) Pub. Date: Nov. 16, 2023

(54) IN VITRO METHOD FOR THE DETECTION OF SARS-COV-2 IN AN ORAL SAMPLE USING A COLORIMETRIC IMMUNOSENSOR AND RELATED COLORIMETRIC IMMUNOSENSOR

(71) Applicant: TECHNOGENETICS S.R.L., Milano (IT)

(72) Inventors: Stefania AMOROSI, Piacenza (IT); Antonio CERVO, Tavazzano con Villavesco (Lodi) (IT); Bartolomeo DELLA VENTURA, Maddaloni (Caserta) (IT); Elena SAUCHELLI, Venticano (Avellino) (IT); Salvatore SESSO, Montella (Avellino) (IT); Raffaele VELOTTA, Napoli (IT)

(21) Appl. No.: 18/246,149

(22) PCT Filed: Sep. 21, 2021
(86) PCT No.: PCT/IB2021/058577

§ 371 Date: Mar. 21, 2023

(30) Foreign Application Priority Data
Sep. 22, 2020 (IT) 102020000022351

(51) Int. Cl. G01N 33/569; G01N 21/78; G01N 21/82; G01N 33/543

(52) U.S. Cl. CPC G01N 33/56983; G01N 21/78; G01N 21/82; G01N 33/54346; G01N 2333/165

(57) ABSTRACT

An in vitro method and a kit for detecting the SARS-CoV-2 virion using a colorimetric immunosensor in an oral biological sample selected from saliva and sputum are provided. The method and kit are based on the use of gold capture nanoparticles carrying on their surface at least one antibody capable of binding a SARS-CoV-2 surface antigen.

### FILED OF THE INVENTION

The present invention relates to an in vitro method for the detection of SARS-CoV-2 in an oral biological sample by using a colorimetric immunosensor and the related colorimetric immunosensor.

### STATE OF THE ART

In the field of Research and Diagnostics, colorimetry-based optical biosensors are becoming increasingly important due to their versatility, ease of use, and ability to reach an extremely low limit of detection (LOD) of the analyte.

Known among the optical biosensors are colorimetric biosensors based on gold nanoparticles, which use a physical property of gold nanoparticles, designated as Localized Surface Plasmon Resonance (LSPR), to monitor the colour change when different-size clusters are formed following the interaction between the analyte to be detected and the gold nanoparticles.

Optical transducers are particularly interesting for the direct detection (label free) of microorganisms. These sensors are designed to detect minimal conversions in refractive index or thickness that occur when cells attach to receptors immobilized on the surface of the transducer, correlating changes in concentration, mass, or number of molecules to direct changes in light characteristics. The sensing mechanism is based on the conversion of signals resulting from binding to the target to be detected into physical signals which can be amplified and detected. Nanomaterials, which are characterized by extremely small dimensions and to which suitable surface modifications can be made, allow highly specific interaction with biomolecular targets, thus showing enormous potential in the field of biological detection.

More generally, gold nanoparticles find application in a wide range of disciplines, including magnetic fluids, catalysis, biotechnology/biomedicine, magnetic resonance imaging, and environmental remediation. In most of these applications, better functionality of these nanoparticles is seen when their size is less than a critical value, which depends on the material but typically is approximately 10-20 nm.

Biosensors based on specificity of interaction between an antigen and the corresponding antibody for determining the analyte of interest are commonly referred to as immunosensors. The antibody immobilization procedure is a crucial step in the construction of these devices since the orientation of the antibody molecules on the electrode surface significantly affects the performance of a biosensor. In fact, the formation of a layer of antibodies with their binding sites well oriented and facing the antigen improves the efficiency of the biosensor, making the choice of the immobilization method one of the most important aspects to be taken into account in the construction of an immunosensor. Generally, antibody immobilization methods involve physical or chemical adsorption of these molecules.

The method using ionic or electrostatic bonds, hydrophobic interactions and van der Waals bonds between the antibody and the surface, and not requiring chemical modifications to the protein (Sharma, Byrne, and Kennedy 2016; Um et al. 2011) is mentioned among the simplest adsorption procedures. The main disadvantage of the above method is that the antibodies are randomly oriented and may therefore not properly expose the antigen binding sites.

More effective methods of immobilization of antibodies are based on the formation of covalent bonds between the antibody and the gold surface (Alves, Kiziltepe, and Bilgicer 2012; Ho et al. 2010; Vashist et al. 2011; Rahman et al. 2007). For example, biotinylated antibodies can be immobilized on surfaces modified with streptavidin or avidin (Barton et al. 2009; Ouerghi et al. 2002) or the antibodies can be immobilized on surfaces modified with proteins such as protein A or protein G (J. E. Lee et al. 2013; Inkpen et al. 2019; Sharafeldin and Rusling 2019; Fowler, Stuart, and Wong 2007). Eventually, methods of immobilization of antibodies involving the trapping in polymer matrices have been developed in the last decade (Sun et al. 2011; Bereli et al. 2013; Moschallski et al. 2013; Yamazoe 2019).

Among the possible immobilization strategies, the formation of self-assembled monolayers (SAMs) is one of the most widely used methods for the construction of immunosensors. For example, the oriented immobilization of an antibody on the gold surface of an electrode can be achieved by exploiting the formation of SAMs of thiol carboxylic acids (Barreiros dos Santos et al. 2013; Malvano, Pilloton, and Albanese 2018; Wan et al. 2016) or by immobilizing the antibodies on electrochemically deposited cysteamine layers (Malvano, Pilloton, and Albanese 2018). In addition, the use of cross-linking agents such as glutaraldehyde, specifically for the immobilization of anti-E. coli antibodies on a polyaniline substrate, has also recently been reported with interesting results in the detection of this bacterium (Chowdhury et al. 2012). Therefore, SAMs are widely used as linkers for the immobilization of antibodies in an oriented manner on a gold surface, but despite the numerous advantages they exhibit in various applications, there are still several aspects that should be taken into consideration in order to understand and control their physical and chemical properties (Vericat et al. 2010; Mandler and Kraus-Ophir 2011; Chaki and Vijayamohanan 2002). A self-assembled monolayer on gold surfaces is commonly represented as a perfect monolayer in which the molecules are in a perfectly packed configuration. Actually, this idea is far from reality, and quality control of a SAM is a crucial point in many applications. The construction of a well assembled monolayer strongly depends on the purity of the chemical reagents and solutions used, and even the presence of a minimum amount of contaminants, such as for example thiolated molecules which are typical impurities in thiol compounds, can lead to a non-uniform and therefore non-ideal layer (C. Y. Lee et al. 2005).

In recent years, different types of immunosensors have been described in studies published in the literature.

Iarossi, M. et al (2018) (“Colorimetric Immunosensor by Aggregation of Photochemically Functionalized Gold Nanoparticles” ACS Omega 3, 4, 3805-3812) describes a colorimetric immunosensor that uses the phenomenon of surface plasmon resonance of gold nanoparticles, as well as the application of this system for the detection of human IgG immunoglobulins.

Liu Y., et al (2015) (“Colorimetric detection of influenza A virus using antibody-functionalized gold nanoparticles” Analyst 140(12)3989-3995) investigated the use of a colorimetric immunosensor based on gold nanoparticles modified with anti-haemagglutinin monoclonal antibodies to determine the influenza A virus. However, no evidence of clinical effectiveness of the immunosensor described is provided in this document.

The research described in Della Ventura B. et al (2020) (“Colorimetric Test for Fast Detection of SARS-CoV-2 in Nasal and Throat Swabs” MedRixv doi: https://doi.org/10.1101/2020.08.15.20175489 and ACS Sensors 5, 3043-3048) concerns the use of a colorimetric immunosensor for the detection of SARS-CoV-2 coronavirus in a nasopharyngeal swab.

The ongoing serious pandemic caused by the novel Severe Acute Respiratory Syndrome Coronavirus 2 (SARS-CoV-2) has posed major public health challenges in many countries. Due to the lack of specificity of symptoms of the serious disease caused by the new coronavirus, called COVID-19, confirmation of diagnosis requires laboratory tests to be performed on respiratory and/or serum samples from patients. Large-scale diagnostic tests also play a key role in isolating asymptomatic COVID-19 patients in an attempt to stem the spread of infection.

Among the procedures currently used for the diagnosis of SARS-CoV-2 coronavirus infection, the method based on reverse transcription polymerase chain reaction (RT-PCR) plays a primary role. This method allows the identification of the viral genome in samples from the upper respiratory tract, in particular in samples taken using nasopharyngeal swabs. However, the diagnostic application of the PCR has major limitations due to the complexity of execution and timing, as well as the need for dedicated instrumentation and trained personnel.

The immunological approaches that have been developed for the diagnosis of COVID-19 also exhibit significant problems. For example, lateral flow assays, while allowing for rapid analysis of many samples, are characterized by low sensitivity.

Further problems associated with the methodologies aimed at diagnosing SARS-CoV-2 infection concern the selection of the most suitable samples to search for viral particles, more specifically intact particles. Among these, the samples of choice are currently represented by respiratory tract samples, in particular nasopharyngeal swabs. However, this sampling requires a precise operating procedure, that is, to be valid, the sampling from the nose must be performed by pushing the swab downwards, to reach the pharynx, and not towards the nasal cavities.

Therefore, there is a need to provide diagnostic tests which allow for rapid identification of the SARS-CoV-2 virus through simple procedures, while maintaining high specificity and sensitivity parameters.

These and other objects are achieved by the in vitro method and the related kit as defined in the attached independent claims, which are suitable for detecting the SARS-CoV-2 virion in an oral biological sample of a subject.

Additional features of the invention are identified in the dependent claims, which form an integral part of the specification.

As will be clear from the following detailed description, the in vitro method according to the invention allows the diagnostic result to be obtained in a very short time, within the minute range, and requires minimum quantities of the sample to be analysed, within a range of approximately one millilitre volume, which are very simple to collect. The particular simplicity of the procedure, which does not require any sophisticated instrumentation, also allows costs to be significantly reduced.

Therefore, a first object of the present invention is an in vitro method for the detection of the SARS-CoV-2 virion in an oral biological sample of a subject selected from saliva and sputum, comprising the steps of:
a) contacting said biological sample with a colloidal suspension of gold capture nanoparticles carrying on their surface at least one antibody capable of binding a SARS-CoV-2 surface antigen, the antigen being selected from the group consisting of the membrane protein (M), envelope protein (E), spike protein (S), and any combination thereof, thereby obtaining a reaction mixture;
(b) determining the formation of a cluster of gold nanoparticles on the surface of the SARS-CoV-2 virion in the reaction mixture, said cluster resulting from the interaction between said antibody and said antigen, the determination being carried out by detecting a change in an optical parameter of the reaction mixture, said change in an optical parameter of the reaction mixture being indicative of the presence of the SARS-CoV-2 virion in the oral sample.

Within the scope of the present description, the term “virion” refers to the mature viral particle, including the genome, nucleocapsid, and envelope thereof.

The method according to the invention is based on the Localized Surface Plasmon Resonance (LSPR) physical principle consisting in the occurrence of coherent and non-propagating oscillations of free electrons in metal particles following irradiation with an electromagnetic wave, the frequency of which resonates with the surface plasmon. The resonance of the surface plasmon, which gives the colloidal solution its colour, depends on various factors, such as the size of the nanoparticles, and can change considerably when the nanoparticles are in contact with each other, or in any case at a distance much smaller than their own diameter. Generally, the coupling of metal nanoparticles, for example gold nanoparticles, to one another in a colloidal suspension occurs through formation of dimers, trimers, or larger chains, up to the formation of clusters, and involves a change in the plasmon resonance, and therefore in the colour of the solution, which can even be detected with the naked eye.

According to the invention, the clustering of the gold nanoparticles occurring in the reaction mixture is mediated by a biological mechanism consisting in the specific interaction between the antibodies immobilized on the surface of said capture nanoparticles and the corresponding antigens occurring on the SARS-CoV-2 virus surface. Therefore, clustering only occurs if the SARS-CoV-2 viral particle is present in the test sample, thus providing extreme specificity to the method of the invention.

Oral samples suitable for use in the method according to the invention are selected from saliva and sputum.

As illustrated in greater detail in the following experimental part, the present inventors surprisingly found that the method of the invention allows detection of the SARS-CoV-2 virus in the saliva or sputum of a subject. Despite the undoubted advantage of the ease of taking this type of samples, to this day their use in a method as previously defined has been particularly difficult, if not impossible, not only due to the high salt concentration in these samples which causes unspecific clustering of the gold nanoparticles, making the biosensor unusable, but also due to the presence of mucus. The latter, while not affecting the kinetics of clustering, contains opaque substances that alter the absorption spectrum. Unfortunately, the variability of the mucosal concentration (not only among people, but also over time for the same person) makes it impossible to standardize the contribution of mucus to the optical reading. In addition, saliva contains a high concentration of proteins that may interfere with the antigen-antibody interaction. All these considerations highlight the peculiarity of the salivary matrix compared to other virion-containing solutions, such as, for example, that consisting of a Virus Transport Medium (VTM). To demonstrate the importance of the peculiarity of each matrix, which makes it almost impossible to immediately extend an analysis technique from one matrix to another, suffice it to think that the “gold standard” technique given by RT-PCR is not effective with the salivary matrix, rather requiring a nasopharyngeal swab with immersion in VTM.

As indicated above, the metal nanoparticle used in the method according to the invention is a gold nanoparticle.

Preferably, the gold nanoparticle has a diameter ranging from 1 nm to 100 nm, more preferably from 2 nm to 40 nm. Most preferred is a gold nanoparticle having a diameter of 20 nm.

In the method according to the invention, the at least one antibody on the surface of the capture gold nanoparticle is capable of binding a SARS-CoV-2 surface antigen selected from the group consisting of the membrane protein (M), envelope protein (E), spike protein (S), and any combination thereof.

As is known in the art, the above-mentioned viral proteins contribute together to the formation of the external viral envelope. Among these, the spike protein is responsible for the binding of SARS-CoV-2 to the host cell by favouring the fusion of the viral envelope with the cell membrane.

Among the antibody molecules suitable for use in the method according to the invention, monoclonal or polyclonal antibodies, monomeric (Fab) or dimeric (F(ab′)2) antibody fragments, single chain antibody fragments (scFv), or any binding protein derived from an antibody scaffold are mentioned by way of non-limiting example.

According to the invention, it is contemplated that anti-SARS-CoV-2 antibodies as defined above may be present on the capture gold nanoparticle, in any possible combination. The proximity of said antibodies to the surface of the gold nanoparticle allows the distance between the nanoparticles of the cluster to be minimal, thus allowing the occurrence of the LSPR phenomenon.

Methods suitable for achieving the immobilization of one or more antibody molecules in the immediate vicinity on the surface of a gold nanoparticle are known in the art, even if they are essentially limited to physisorption or to a photochemical technique (Photochemical Immobilization Technique, PIT) in which the immobilization of the antibodies on the gold surface in the correct orientation is achieved by irradiating these molecules with UV light [Della Ventura B. et al. (2019) “Biosensor surface functionalization by a simple photochemical immobilization of antibodies: experimental characterization by mass spectrometry and surface enhanced Raman spectroscopy”. Analyst 144, 6871-6880].

Unlike physisorption, the PIT method allows a robust and durable functionalization, allowing industrial application of the method.

The selection of the most appropriate antibody immobilization method for use within the scope of the present invention falls well within the skills of those of ordinary skill in the art.

Preferably, the amount of gold capture nanoparticles as defined above in the colloidal suspension is in the range of 1 to 10²⁰ nanoparticles(np)/ml based on the total volume of the suspension, more preferably 10 to 10¹⁵ np/ml. In an even more preferred embodiment, the amount of gold capture nanoparticles is 10¹⁰ np/ml based on the total volume of the suspension.

Advantageously, the method according to the invention allows the SARS-CoV-2 viral particle to be detected in its entirety in the test sample due to the ability of the gold nanoparticles carrying antibodies specifically directed against the viral surface proteins to cluster on the surface of the virion by keeping very close to each other and forming a layer around it. Therefore, thanks to the specific determination of active viral particles, the method according to the invention is particularly suitable for identifying cases in which a SARS-CoV-2 infection is still ongoing.

According to one embodiment, the method of the invention further comprises the step of filtering the reaction mixture obtained in step a) by using a filter element having pores with a diameter ranging from 35 to 65 microns (μm), preferably from 40 to 60 μm, more preferably from 45 to 55 μm, for example a diameter of 45, 46, 47, 48, 49, 50, 51, 52, 53, 54 or 55 μm.

Within the scope of the invention, the above filtration step removes or significantly reduces the mucosal component of the saliva or sputum samples present in the reaction mixture, thereby increasing the sensitivity and specificity of the method of the invention.

As illustrated above, in the method according to the invention the clustering of gold nanoparticles on the surface of the SARS-CoV-2 virion is determined by detecting a change in an optical parameter of the reaction mixture.

In one embodiment of the method of the invention, the detected change in the optical parameter is a colour change of the reaction mixture that is detectable by the naked eye.

In this embodiment, a further optional step consists in comparing the detected colour of the reaction mixture with a colorimetric scale. This step increases the interpretative quality of the result.

Also importantly and advantageously, the embodiment illustrated above does not require the use of instrumentation.

In another embodiment of the invention, the detected change in the optical parameter is a reduction in the transmittance value of the reaction mixture measured at a predetermined wavelength in the visible range, preferably at 560 nm.

Within the scope of the present description, the expression “wavelength in the visible range” refers to a wavelength between approximately 390 nm and approximately 760 nm.

According to the above embodiment, the measurement of the transmittance value of the reaction mixture can be carried out by using a photometer or colorimeter instrument, preferably calibrated with a standard solution having a transmittance value of 100%.

In yet another embodiment of the invention, the detected change in the optical parameter is an increase in the absorbance value of the reaction mixture measured at a predetermined wavelength in the visible range, preferably at 560 nm. Suitable instrumentation for carrying out the above absorbance measurement is a spectrophotometer, for example, which can be a portable or benchtop spectrophotometer.

According to a further embodiment, the detected change in the optical parameter is an increase in the area under the absorption spectrum of the reaction mixture in a wavelength range between 200 nm and 700 nm.

In this embodiment, the method according to the invention allows a quantitative measurement which is indicative of the SARS-CoV-2 viral load in the test sample. According to this embodiment, the use of a standard curve in addition makes it possible to obtain an “absolute” measure of the viral load.

In yet another embodiment, the method of the invention is in a competitive format, wherein the change in the optical parameter is an increase in the absorbance value of the reaction mixture measured at a wavelength in a range from 600 nm to 700 nm.

This competitive embodiment involves the use of a high salt, and therefore particularly unstable colloidal suspension of gold capture nanoparticles. More specifically, according to this embodiment, the colloidal suspension comprises a salt selected from the group consisting of sodium citrate, sodium chloride, potassium phosphate, sodium phosphate, calcium chloride, potassium chloride, and any combination thereof, and the above salt is present in the colloidal suspension at a concentration in the range from 150 milliMolar (mM) to 250 mM.

Within the scope of the invention, preferably the salt is sodium citrate, more preferably sodium citrate present in the colloidal suspension at a concentration of 160 mM.

The presence of the salts causes the addition of saliva free of, and therefore not infected with the SARS-CoV-2 virus to induce the aggregation of the gold capture nanoparticles with one another in the form of a cluster, resulting in a colour change that can be seen with the naked eye in the reaction mixture. If, on the other hand, the saliva contains SARS-CoV-2 viral particles, the formation of clusters of functionalized gold nanoparticles on the surface of the virion prevents said nanoparticles from clustering with each other. Since the clustering of the gold nanoparticles on the surface of the virion causes a shift of the resonance peak in the absorption spectrum of the reaction mixture different from that caused by the clustering induced by salts in the absence of viral particles, more precisely, from a wavelength of approximately 560 nm to a wavelength in the range from 600 to 700 nm, the presence of viral particles in the oral sample involves a slight change in colour compared to the very bright colour change observed in the absence of the virion.

As previously stated, a kit including means suitable to perform the method according to the invention is also included within the scope of the present invention.

Therefore, a second aspect of the present invention is a diagnostic kit for the detection of the SARS-CoV-2 virion in an oral biological sample of a subject selected from saliva and sputum, comprising a colloidal suspension of gold capture nanoparticles carrying on their surface at least one antibody capable of binding a SARS-CoV-2 surface antigen, the antigen being selected from the group consisting of the membrane protein (M), envelope protein (E), spike protein (S), and any combination thereof.

According to one embodiment, the colloidal mixture of gold capture nanoparticles comprises a salt selected from the group consisting of sodium citrate, sodium chloride, potassium phosphate, sodium phosphate, calcium chloride, potassium chloride, and any combination thereof, said salt being present in the colloidal suspension at a concentration in the range from 150 milliMolar (mM) to 250 mM.

Within the scope of the invention, preferably the salt is sodium citrate, more preferably sodium citrate present in the colloidal suspension at a concentration of 160 mM.

In one embodiment, the diagnostic kit of the invention also comprises a support containing a colorimetric scale, for example a colorimetric strip.

In another embodiment, the diagnostic kit of the invention also comprises a portable colorimeter or photometer.

Preferably, the portable photometer is equipped with a tungsten lamp and a monochromator capable of isolating the wavelength at 560 nm.

Preferably, the portable colorimeter is equipped with a diode capable of emission at 560 nm.

Among the portable instruments suitable for use in the kit of the invention, the portable model HI96759 photometer and the portable model HI759 colorimeter, both from Hanna Instruments, are mentioned as examples.

In the diagnostic kit of the invention, the colloidal suspension comprising the gold capture nanoparticles can be dispensed into a plurality of single disposable test tubes.

Alternatively, said colloidal suspension can be supplied in a single package, for example in a dedicated dropper device.

In a further embodiment, the diagnostic kit of the invention also comprises a filter element as previously defined in connection with the method of the invention.

According to this embodiment, the filter element can be housed inside a disposable test tube, preferably in the vicinity of one end of the body of the test tube opposite the end where the colloidal suspension is introduced. Within the scope of the invention, the filter element in the kit is preferably a hydrophilic polyethylene filter.

The following experimental examples are provided for illustrative purposes only. Therein, reference is made to the accompanying drawings, wherein:

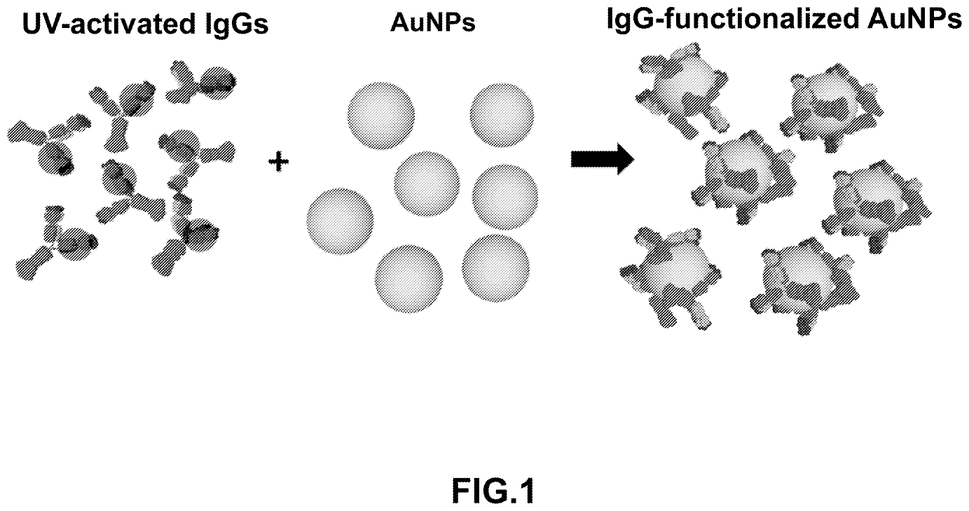

FIG. 1 is a schematic representation of the method for functionalizing the surface of gold nanoparticles with type G immunoglobulins (Photochemical Immobilization Technique, PIT). IgG antibodies are irradiated with UV rays using a lamp of appropriate power, causing the reduction of disulfide bridges at specific positions in the light chain constant part of the antibody. The production of thiols allows the formation of a covalent bond between the antibody and the surface of the gold nanoparticle, leaving one of the two antigen-recognition portions of the antibody free.

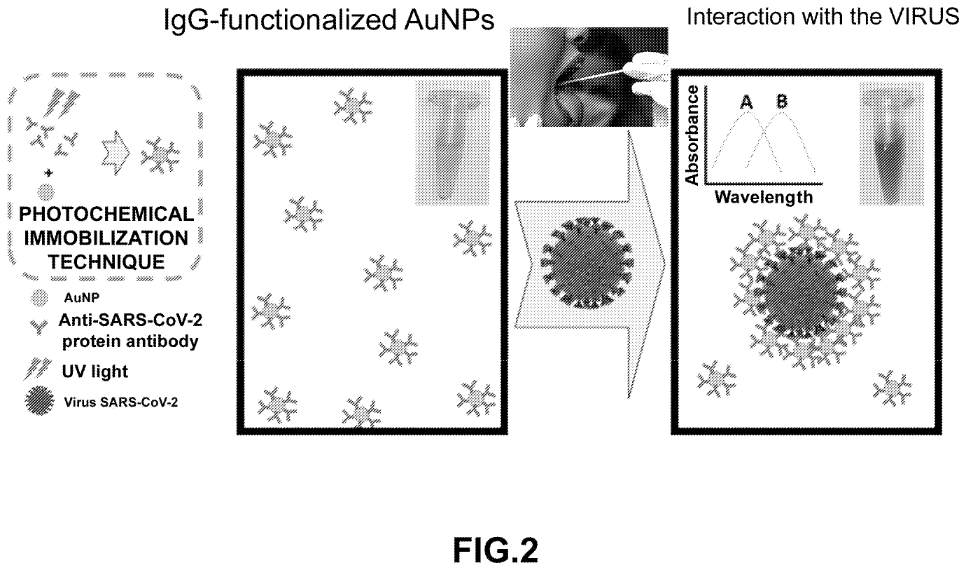

FIG. 2 is a schematic representation of the method of the invention. The colloidal suspension of gold nanoparticles functionalized with anti-SARS-CoV-2 antibodies is contacted with the sample containing the virus, thus forming a reaction mixture. Following the clustering of the functionalized gold nanoparticles around the viral particle, the reaction mixture changes colour. As the concentration of viral particles increases, the shift towards blue increases.

### EXPERIMENTAL SECTION

### Example 1: Preparation of the Colloidal Gold Solution (Synthesis of Nanoparticles)

For their experiments, the present inventors obtained the synthesis of gold nanoparticles having a diameter of approximately 20 nm using a variant of a protocol known in the art (the Turkevich method). According to this protocol, tetrachloroauric acid is first solubilized in water and the addition of sodium citrate causes reduction of the gold, resulting in the production of a gold seed and subsequently the growth of gold around it. The synthesis reaction consisted in mixing 1 mL of HAuCl4 (10 mg/mL) and 2 mL of sodium citrate dihydrate (25 mg/mL) in 100 mL of milliQ (ultrapure) water. The operating temperature was maintained at 90° C., with gentle stirring. The formation of gold nanoparticles was identified by a drastic change in the colour of the solution from yellow to orange.

At the end of the synthesis, the solution was centrifuged at 6 G for 30 minutes, thereby obtaining gold nanoparticles ready to be functionalized.

### Example 2: Functionalization

The surface of the gold nanoparticles was functionalized by using the mechanism known as Photochemical Immobilization Technique (PIT), as described in FIG. 1.

Briefly, IgG antibodies directed against the membrane protein (Membrane, M), the envelope protein (Envelope, E), and the spike protein (S) of the SARS-CoV-2 virus were used (0.1 mg/mL).

A quartz cuvette containing the antibody solution at a concentration of 1 μg/mL was inserted into a specially designed UV lamp and irradiated with UV rays for 30 seconds in order to obtain the reduction of some disulfide bridges in specific positions of the antibody. Subsequently, gold nanoparticles having a diameter of 20 nm in size were functionalized, obtaining a concentration of nanoparticles with the anti-envelope antibody of 10¹⁰ nanoparticles (np)/mL, a concentration of nanoparticles with the anti-spike antibody of 10¹⁰ np/mL, and a concentration of nanoparticles with the anti-membrane antibody of 10¹⁰ np/mL. Any empty spaces left on the gold nanoparticles were then blocked by using a solution containing BSA (50 μg/mL).

Finally, the colloidal suspensions containing the three different antibodies were mixed together in a ratio of 1:1:1 so as to obtain a single suspension of gold nanoparticles carrying the three anti-SARS-CoV-2 antibodies, thus significantly increasing the specificity of the system.

Purification of the obtained samples was carried out by centrifugation at 6 G for 10 minutes.

### Example 3: Sample Preparation

The saliva sample was collected using a swab and immediately transferred into a volume of 0.5 ml of the colloidal suspension of functionalized gold nanoparticles. Any virion on the swab was released by vigorously rotating the swab on its own axis for about 10 seconds in the suspension. The present inventors found that, unlike the prior art assays, the method according to the invention surprisingly does not require the saliva sample to be resuspended in a buffer solution after the sampling. The present inventors also found that other stirring methods, for example stirring the test tube up and down in an uncoordinated manner, do not achieve the same result in the same times, also showing lack of repeatability and reproducibility.

Optionally, in order to remove the mucosal component present in the samples, the reaction mixture obtained by mixing the saliva or sputum samples with the colloidal suspension of the gold capture nanoparticles was allowed to drip through a hydrophilic polyethylene filter with pores of 50 microns in diameter and collected in an underlying cuvette. The time taken for this passage varied over a 3-10-minute interval according to the viscosity of the saliva. After the filtration, the reaction mixture was read.

### Example 4: Detection

Gold capture nanoparticles prepared and functionalized as described above were used for the SARS-CoV-2 virus detection experiments.

Briefly, after mixing the saliva or sputum sample with the colloidal suspension of the gold capture nanoparticles, a colour change occurred in the reaction mixture that went from orange to blue passing through purple. In particular, the shift towards blue increased with increasing concentration of virus. The simple buffer solution in which no colour change was seen was used as a negative control.

For transmittance or absorbance analysis, a defined volume of the saliva or sputum sample, after being resuspended, was deposited by a sterile disposable Pasteur pipette in a test tube (Wheaton type) already containing the gold capture nanoparticle suspension. Alternatively, defined volumes of the test sample and the gold capture nanoparticle suspension were dispensed together into a supplied cuvette.

The reaction mixture obtained with the preparation procedures described above was mixed by stirring the test tube/cuvette, or by pipetting, thereby allowing the formation of the clusters of functionalized gold nanoparticles on the surface of the SARS-CoV-2 virion.

Subsequently, and optionally after filtering, the test tube or cuvette was housed in the supplied portable reader, which provided absorbance or transmittance values indicative of the positivity/negativity of the sample, i.e., the presence or absence of SARS-CoV-2 viral particles.

In the experiments carried out by the present inventors, a portable photometer was used, which was equipped with a tungsten lamp and a monochromator capable of isolating the wavelength at 560 nm, after suitable calibration with a standard. A simple “blank”, i.e., a sample assigned a 100% transmittance or 0% absorbance, was used as the standard. Disposable cuvettes containing the reaction mixture were used for the photometric analysis.

In their experiments, the present inventors alternatively used a colorimeter device equipped with a diode capable of emitting at 560 nm and, similarly to the above-mentioned device, of returning a transmittance value. In this procedural mode, after being resuspended, the saliva or sputum sample was dispensed and assayed directly in the disposable reaction tube pre-packaged with the colloidal suspension of gold capture nanoparticles.

The transmittance values measured in the experiments described above are indicative of the amount of SARS-CoV-2 viral particles present in the test sample.

In order to measure the absorbance of the reaction mixture, the inventors also used a benchtop spectrophotometer instrument which is capable of emitting in a wide spectrum of wavelengths ranging, for example, from 200 to 700 nm. The absorbance measurements taken at the different wavelengths allowed the area under the absorption spectrum to be calculated, thus providing a “relative” quantitative determination of the viral load. The viral load measurement can become “absolute” by means of a calibration of the described technique.

Table 1 below shows the results obtained in 6 patients (3 positive and 3 negative) using the PCR method and the method according to the invention in parallel. For the PCR analysis, the data is expressed as the cycle threshold (Ct), which corresponds to the PCR reaction cycle in which the emitted fluorescence exceeds the threshold. For the analysis according to the method of the invention, the data is expressed as absorbance values (Abs). The term “x” represents a negative sample.

TABLE 1

| PCR threshold (Ct) | Abs@560 nm |
|---|---|
| 18 | 0.325 |
| 22 | 0.294 |
| 35 | 0.224 |
| x | 0.171 |
| x | 0.195 |
| x | 0.176 |

Table 1 shows that negative samples give an absorbance value of 0.18±0.01. Even considering the 3 SD criterion, the sample with the lowest viral load (Ct=35) is clearly distinguished from the negative samples. In fact, values higher than 35 for the PCR threshold are considered negative. These results demonstrate that the method according to the invention has a detection limit comparable to the PCR.

### Example 5: Method of the Invention with a Competitive Configuration

For the experiments carried out using the method of the invention with a competitive configuration, a particularly unstable colloidal suspension of capture nanoparticles was produced. To this end, the following two steps were carried out: 1) a 160 mM concentration of sodium citrate was used during the step of production in water; 2) the excess reagents were removed by centrifuging at 4.5 G for 30 minutes, and the precipitate was resuspended in an aqueous solution containing 160 mM sodium citrate. Subsequently, the addition of saliva containing SARS-CoV-2 viral particles to the colloidal suspension of capture nanoparticles obtained as described above caused a shift of the resonance peak in the absorption spectrum of the reaction mixture different from that caused by the reaction mixture to which uninfected saliva was added and in which, therefore, the clustering of the nanoparticles was solely induced by salts. In fact, the present inventors observed a very bright colour change of the reaction mixture in the absence of the virion, whereas they witnessed a slight colour change in the presence of the virus.

In the method of the invention with the direct configuration, as described in Example 4, an absorbance peak of the reaction mixture is observed at a wavelength of approximately 560 nm, whereas in the method of the invention with the competitive configuration, the peak of the reaction mixture, which is much larger, is observed at a wavelength in the visible range of 600-700 nm.

Table 2 below shows the results obtained in 6 patients (3 positive and 3 negative) using the PCR method and the method according to the invention in parallel. The data is expressed as indicated above with reference to Table 1. The term “x” represents a negative sample.

TABLE 2

| PCR threshold (Ct) | Abs@560 nm | Abs@600 nm |
|---|---|---|
| 15 | 0.274 | 0.157 |
| 19 | 0.252 | 0.133 |
| 22 | 0.214 | 0.122 |
| x | 0.199 | 0.046 |
| x | 0.187 | 0.052 |
| x | 0.192 | 0.064 |

The results shown in Table 2 demonstrate the presence of measurable differences between samples containing SARS-CoV-2 (positive cases) and virus-free samples (negative cases).

What is claimed is:

1. An in vitro method for detecting the SARS-CoV-2 virion in an oral biological sample of a subject selected from saliva and sputum, the method comprising:
a) contacting said oral biological sample with a colloidal suspension of gold capture nanoparticles carrying on their surface at least one antibody capable of binding a SARS-CoV-2 surface antigen, the SARS-CoV-2 surface antigen being selected from the group consisting of membrane protein (M), envelope protein (E), spike protein (S), and any combination thereof, thereby obtaining a reaction mixture; and
(b) determining the formation of a cluster of the gold capture nanoparticles on the surface of the SARS-CoV-2 virion in the reaction mixture, said cluster resulting from an interaction between said at least one antibody and said SARS-CoV-2 surface antigen, the determination being carried out by detecting a change in an optical parameter of the reaction mixture, said change in the optical parameter of the reaction mixture being indicative of the presence of the SARS-CoV-2 virion in the oral biological sample.

2. The in vitro method of claim 1, wherein the change in the optical parameter is a colour change of the reaction mixture detectable by the naked eye.

3. The in vitro method of claim 2, further comprising comparing the detected colour of the reaction mixture with a colorimetric scale.

4. The in vitro method of claim 1, wherein the change in the optical parameter is a reduction in the transmittance value of the reaction mixture measured at a predetermined wavelength in the visible range.

5. The in vitro method of claim 1, wherein the change in the optical parameter is an increase in the absorbance value of the reaction mixture measured at a predetermined wavelength in the visible range.

6. The in vitro method of claim 1, wherein the change in the optical parameter is an increase in the area under the absorption spectrum of the reaction mixture in a wavelength range between 200 nm and 700 nm.

7. The in vitro method of claim 1, wherein the in vitro method is in a competitive format,
wherein the colloidal suspension of the gold capture nanoparticles comprises a salt selected from the group consisting of sodium citrate, sodium chloride, potassium phosphate, sodium phosphate, calcium chloride, potassium chloride, and any combination thereof, said salt being present in the colloidal suspension at a concentration ranging from 150 milliMolar (mM) to 250 mM, and
wherein the change in the optical parameter is an increase in the absorbance value of the reaction mixture measured at a wavelength in a range from 600 nm to 700 nm.

8. The in vitro method of claim 1, further comprising filtering the reaction mixture obtained in step a) by using a filter element having pores with a diameter ranging from 35 to 65 microns (μm).

9. The in vitro method of claim 1, wherein the change in the optical parameter is detected by a colorimeter or a photometer.

10. A diagnostic kit for detecting the SARS-CoV-2 virion in an oral biological sample of a subject selected from saliva and sputum, comprising a colloidal suspension of gold capture nanoparticles carrying on their surface at least one antibody capable of binding a SARS-CoV-2 surface antigen, the SARS-CoV-2 surface antigen being selected from the group consisting of membrane protein (M), envelope protein (E), spike protein (S), and any combination thereof.

11. The diagnostic kit of claim 10, wherein the colloidal suspension comprises a salt selected from the group consisting of sodium citrate, sodium chloride, potassium phosphate, sodium phosphate, calcium chloride, potassium chloride, and any combination thereof, said salt being present in the colloidal suspension at a concentration ranging from 150 milliMolar (mM) to 250 mM.

12. The diagnostic kit of claim 10, wherein the colloidal suspension is dispensed into a plurality of single disposable test tubes.

13. The diagnostic kit of claim 10, further comprising a support containing a colorimetric scale.

14. The diagnostic kit of claim 10, further comprising a portable colorimeter or a photometer.

15. The diagnostic kit of claim 10, further comprising a filter element having pores with a diameter ranging from 35 to 65 microns (μm).

16. The diagnostic kit of claim 15, wherein the filter element is a hydrophilic polyethylene filter.

17. The in vitro method of claim 4, wherein the change in the optical parameter is a reduction in the transmittance value of the reaction mixture measured at 560 nm.

18. The in vitro method of claim 5, wherein the change in the optical parameter is an increase in the absorbance value of the reaction mixture measured at 560 nm.
